(19) United States
(12) Patent Application Publication
(10) Pub. No.: US 2018/0116867 A1
(43) Pub. Date: May 3, 2018

(54) MASSAGING DEVICE HAVING TWO OPPOSITE MASSAGING SURFACES FOR MASSAGING HUMAN BODY

(71) Applicant: Kun Yuan Tong, Suwanee, GA (US)

(72) Inventor: Kun Yuan Tong, Suwanee, GA (US)

(21) Appl. No.: 15/853,580

(22) Filed: Dec. 22, 2017

Related U.S. Application Data
(63) Continuation-in-part of application No. 14/121,489, filed on Sep. 11, 2014, now abandoned.

(51) Int. Cl. A61F 7/03; A61H 15/00; A61H 15/02

(52) U.S. Cl. CPC A61F 7/03; A61H 15/0078; A61F 2007/0001; A61H 2201/1669; A61H 2201/1678; A61H 15/02

(57) ABSTRACT

The present invention provides a massaging device having two opposite massaging surfaces for massaging human body. A heating unit is encapsulated inside a casing, and the casing includes two parts that are releasably connected to each other, each part having a truncated shape of oblate spheroid. The invention exhibits numerous technical merits such as simpler but robust structure, improved manufacturability, and capability of maintaining warm temperature for longer period of time, among others.

### CROSS-REFERENCE TO RELATED APPLICATIONS

This non-provisional application is a Continuation-in-Part of U.S. application Ser. No. 14/121,489, filed on Sep. 11, 2014, the entire disclosure of which is incorporated herein by reference.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### FIELD OF THE INVENTION

The present invention generally relates to a massaging device having two opposite massaging surfaces for massaging human body.

### BACKGROUND OF THE INVENTION

A problem with hot massage stone is that it will cool off in about two minutes and needs to be reheated by hot water again. There is a need to develop a massaging device that has a simpler but robust structure, that is easy to manufacture, and that can maintain a warm temperature for a long period of time.

U.S. Pat. No. 2,638,527 to Curtis Cecil Claud has disclosed a massage and heat applicator as shown in FIGS. 1-4. FIG. 1 is a longitudinal central section of the implement containing a removable heating unit. FIG. 2 is a similar longitudinal section on a line at right angles to that of FIG. 1. FIG. 3 is a longitudinal sectional view of the removable heating unit detached from the implement. FIG. 4 is a fragmentary sectional view of the implement provided with a combined heating coil and thermostat in lieu of the heating lamp element of the other figures. Reference number 5 designates the tubular or hollow handle or grip portion of the implement by which the same may be manipulated with respect to the ailing part to be treated, the same having a threaded extension 6 adapted to receive the complementary collar 7 of the hollow head 8, the interior of which opens to the hollow handle or grip. The grip 5 and head 8 are formed of metal such as steel or aluminum and the head 8 has the laterally extended branches 9 and 10 provided with small escape apertures 11 to facilitate circulation of air through the hollow head.

The branches 9 and 10 of the head are curved to have the configuration shown in. FIGS. 1 and 2, so that the operating or contact face 12 thereof is concaved toward the handle 5 in order to combine with the curvature generally present in a portion of the body to be treated. To avoid any harsh contact or impediment to'the manipulation of the implement over the skin of the patient, the head 8 throughout its extent is rounded transversely at the ends thereof to eliminate corners or edges As apparent from. FIG. 2, 13 denoting the curvature or the outer surface of the arms and 14 the surface of the intermediate or connecting portion of the arms.

On the interior of the grip or handle 5 there is a shoulder 15 facing that end 16 of the grip opposite to the headed end thereof. The opening 17 through the grip or handle being of slightly greater and uniform diameter leading to the shoulder than that portion of the opening 18 of uniform, but slightly reduced diameter, constitutes a receiving chamber for the reception of a heating element or unit now to be defined. The unit comprises a cylinder 19 of insulating material, with insulated wire terminal supporting means 20 inserted in one end (FIG. 3), the insulated wiring being shown at 21 and 22, leading to an ordinary or preferred incandescent lamp 23, the latter having the customary stem 24 screwed into a metal thimble 25, carried by an insulated cap 26 at the end of the cylinder 19. The near ends of the wires 21 and 22 are in the customary electrical communication with the filament of the lamp or bulb 23 as at exposed or uninsulated portions 21^{a} and 22^{a} of the otherwise insulated wires 21 and 22. In inserting this unit just described in the grip or handle 5, the lamp bulb 23 may be removed and the balance of the unit intact inserted through the bottom of the handle when the lamp bulb 23 may be restored to the unit, the head 8 being, removed temporarily for that purpose. When the bulb is relatively smaller than that illustrated, the same may be attached to the unit before it is inserted in the grip or handle. When the heating unit is seated in the handle the same is held in place by a clamping ring 27 having threaded engagement with the end 16 of the grip or handle, which will effect a seating of the flange 28 for the unit against the shoulder 15 of the handle 5. In lieu of the light bulb 23, it may at times employ the resistance coil 29, illustrated in FIG. 4, the operation of which is well understood in the art, the same being a resistance wire introduced in the electrical circuit and wound or supported upon a core of suitable substance, such as porcelain 30. To enable the operator to know that the implement is in heating state, a small disc of transparent plastic material 31 is screwed into one side wall of the head, as at 32, and by this means the illumination of the bulb 23 or glow from the coil 29 may be seen.

At times it may be desirable to have a thermostat associated with the heating unit to prevent extreme heat imparted by the lamp or coil to the threaded head 8 or to constantly maintain a given thermal temperature of the head. To these ends the thermostat 33 (FIG. 3) is provided. In the instance of the device of FIG. 4, the porcelain core 30, on which the resistance wire 29 is coiled, is hollow and the thermostat 33 is carried on the interior thereof, circulating openings 34 being formed for air passage through the hollow core or shell

US Patent Application Publication 2008/0168605 to Wolske discloses a thermal pillow apparatus comprising a pillow element, a heat exchanger, a thermal liquid, a fluid pump and a controller. The thermal liquid circulates between the heat exchanger and the pillow element in a closed loop, transferring thermal energy between the heat exchanger and the pillow element. A fluid pump is disposed in the closed loop to aid in circulating the thermal liquid within the closed loop. A Peltier device is the typical heat pump element used in the heat exchanger. The controller coordinates the operation of the thermal pillow apparatus, for example monitoring the temperature of the thermal liquid, and activating the heat exchanger and the fluid pump. FIG. 5 is a schematic view of the thermal pillow apparatus. FIG. 6 is an isolated schematic view of a pillow element of the thermal pillow apparatus of FIG. 5. FIG. 7A is a sectional view of a pillow element of the thermal pillow apparatus of FIG. 5. FIG. 7B is a sectional view of another pillow element of the thermal pillow apparatus of FIG. 5.

The thermal apparatus 100 in FIG. 5 comprises a pillow element 102, a heat exchanger 104, a controller 106, a fluid pump 108, a control pendant 110, and a power supply 112. In FIGS. 5, 6, 7A and 7B, pillow element 102 comprises a cushion element 122, a bladder 114, an outer casing 116, a pillow element thermal liquid inlet 118 and a pillow element thermal liquid outlet 120. Pillow element 102 typically has an outer perimeter defined by the outer casing 116. The outer casing 116 may be made of material such as, for example, cotton, polyester, foam, memory foam or any fabric or material. The outer casing 116 may be constructed as a sleeve that is sized to contain the bladder 114 and the cushion element 122. Alternatively, the outer casing 116 may be defined by the outer perimeter of the cushion element 122, or the outer casing 116 may be defined by a bladder outer wall element 130. A user may directly tactilely interact with the outer casing 116, for example by placing their head on the outer casing 116 when they are sleeping, or by placing the outer casing 116 adjacent to an area of chronic pain, such as, for example a lower back. In addition, the outer casing 116 also typically comprises openings (not shown) adapted for the pillow element thermal liquid inlet 118, and the pillow element thermal liquid outlet 120. The openings (not shown) are usually dimensioned so that the pillow element thermal liquid inlet 118 and the pillow element thermal liquid outlet 120 can pass through the openings (not shown), permitting the pillow element thermal liquid inlet 118 and the pillow element thermal liquid outlet 120 to be in fluid communication with a first thermal liquid circulation conduit 124, and a second thermal liquid circulation conduit 136, respectively. The cushion element 122 is located between the outer casing 116 and the bladder 114. The cushion element 122 may be composed of cotton batting, memory foam, open celled foam, closed cell foam, any mixture of the above. The pillow element 102 may not comprise a cushion element 122. For example, the pillow element 102 may comprise a bladder 114, an outer casing 116, a pillow element thermal liquid inlet 118 and a pillow element thermal liquid outlet 120. The bladder 114 typically comprises a bladder outer wall 130, a first bladder chamber 126, and a second bladder chamber 128. The bladder outer wall 130 may be, for example, constructed of a liquid impermeable flexible material such as plastic or rubber suitable for use with the thermal liquid 132. The bladder outlet wall 130 can deform as pressure is applied. For example, if a user places a load, such as for example their head, onto the pillow element 102, there may be a pressure applied to the bladder outer wall 130 causing the bladder outer wall 130 to deform in response to the pressure. Alternatively, a pressure applied to the interior of the bladder outer wall 130, from, for example, a hydrostatic pressure from the thermal liquid 132, may also cause the bladder outer wall 130 to deform The bladder 114 comprises a first bladder chamber 126. Typically the first bladder chamber 126 is in fluid communication with the first and second thermal liquid circulation conduits 124, 136 through the pillow element thermal liquid inlet 118 and the pillow element thermal liquid outlet 120. Typically, the first bladder chamber 126 contains at least some thermal liquid 132 The first bladder chamber 126, aside from the pillow element thermal liquid inlet 118 and the pillow element thermal liquid outlet 120, is typically sealed such that the thermal liquid 132 cannot leak out. In another example, the first bladder chamber 126 may include a resealable inlet (not shown) that permits a user to add, remove, or alter the thermal liquid 132 in the first bladder chamber 126. Thermal liquid 132 may be circulated from a heat exchanger 104 to the first bladder chamber 126 of the bladder 114 within pillow element 102 via the first thermal liquid circulation conduit 124 From the first bladder chamber 126, the thermal liquid 132 typically transfers thermal energy from or to a user who is adjacent to the pillow element 102. The thermal liquid 132 is then typically re-circulated to the heat exchanger 104 via a second thermal liquid circulation conduit 136. As such, the thermal liquid 132 operates in a closed loop between the heat exchanger 104 and the pillow element 102. A fluid pump 108, that is located within the closed loop in which the thermal liquid 132 circulates, typically circulates the thermal liquid 132 within the closed loop. In another example, the first bladder chamber 126 may also comprise a plurality of galleries 134. The galleries 134 may aid in directing and forcing circulation of the thermal liquid 132 within the first bladder chamber 126. The galleries 134 may, for example, aid in ensuring a more uniform circulation of thermal liquid 132 in the first bladder chamber 126. The galleries 134 may also therefore ensure a more even transfer of thermal energy to and from the thermal liquid 132 located within the first bladder chamber 126.

The bladder 114 may also comprise a second bladder chamber 128. The second bladder chamber 128 is thermally coupled to the first bladder chamber 126. As illustrated in FIGS. 7A and 7B, the second bladder chamber 128 may be located adjacent to only one side of the first bladder chamber (FIG. 7A), or the second bladder element 128 may be located adjacent to both sides of the bladder chamber 128 (FIG. 7B). The second bladder chamber 128 is contained within the bladder outer wall 130 However the second bladder chamber 128 is separated from the first bladder chamber 126 by a bladder inner wall 138. The bladder inner wall 138 is therefore adjacent to both the first bladder chamber 126 and'the second bladder chamber 128. In FIG. 7A, the bladder has a three-ply construction comprised of the top bladder outer wall 130, the bladder inner wall 138, and bottom bladder outer wall 130. At the outer edge of the bladder 114, the three-ply construction of the bladder 114 may have common specific bonding paths. In an embodiment, the second bladder chamber 128 comprises a gel 140 The gel 140 may be a viscous fluid, or any other gelatinous material. The gel 140 may aid in regulating, for example evening out, the thermal profile of the thermal energy being transferred to or from the thermal liquid 132 in the first bladder chamber 126. The gel 140 may therefore, for example, keep the pillow element 102 from having portions that are of significantly different temperatures. The gel 140 may also aid in sustaining an even flow of thermal energy to and from the thermal liquid 132 in the first bladder chamber 126, even as the thermal characteristics of the thermal liquid 132 change. In FIG. 6, a thermal load may be applied to the pillow element 102. The thermal load is typically the user, for example a user's head, being applied to the pillow element 102. The placement of a user's head introduces a source or sink of thermal energy to or from the pillow apparatus 102 The thermal source may thereafter transfer or receive thermal energy from the pillow element 102, through the thermal energy transferred from the thermal liquid 132 through the bladder 114, the cushion element 122, and the outer casing 116 of the pillow element 102. Typically, the thermal load, such as the user's head or body part, is applied to the side of the pillow element 102 that is adjacent to the second bladder chamber 128. This may be only one side of the pillow element 102, as shown in FIG. 7A, or both sides of the pillow element 102 shown in FIG. 7B.

U.S. Pat. No. 2,803,243 to Karoline discloses a stroking-massage apparatus. FIGS. 8 and 9 show axial sections of a constructional form of the stroking-massage apparatus on planes perpendicular to one another. Casing 1 consisting of Bakelite bears at its base a socket 2 into which the plug-sockets 3 and 4 are pressed, which are separated spark-proof by a rib of the base-socket. In between them and the screwed plug-sockets 5 and 6 which are inserted into them contact blades 7 and 8 are clamped; the leads-in 9 and 10 of a cable 11 which leave the casing through the ring 12 are connected to these contact blades. The plugs 13, 14 of the holder 15 of a filament lamp 16 are inserted into the sockets 3, 4 or into the screwed sockets 5, 6 until the lamp rests on the insulating rib. The base of the casing is pierced within this rib by an axial opening (or bore) 17. The filament lamp is surrounded by a jacket 18; this jacket has an angular slot 19 catching with the pin 20 of the casing. The jacket 18 has at its outer end a screw-thread 21 serving for the screwing on of a cap 22 of blue synthetic resin. By an outer, concave face 23 and an inner, somewhat more bent face 24 the front of this cap is shaped in the way of a convexo-concave lens. Each face of the lens has varying radii of curvature. Between casing 1 and jacket 18 or cap 22 is placed a packing ring 25. The jacket together with the cap may easily be connected to the casing or separated therefrom by means of the bayonet-lock-like fastening consisting of the slot 19 and the pin 20. By screwing the cap 22 on the jacket 18 a focal adjustment of the lens consisting of the faces 23 and 24 is made possible; thereby the apparatus may be used either as a point-irradiating device for deep-treatment, or as a surface-irradiating device with a greater dispersive action.

However, none of the prior art technology can provide a massaging device that has a simpler but robust structure, that is easy to manufacture, and that can maintain a warm temperature for a long period of time. Advantageously, the present invention can overcome the aforementioned problems.

### SUMMARY OF THE INVENTION

The present invention provides a massaging device having two opposite massaging surfaces for massaging human body The device includes a casing and a heating unit encapsulated inside the casing. The casing comprises a first part including a truncated shape of a first oblate spheroid and a second part including a truncated shape of a second oblate spheroid. The truncated shape of the first oblate spheroid is formed by cutting away a portion of the first oblate spheroid along a plane perpendicular to a minor axis of the first oblate spheroid. The truncated shape of the second oblate spheroid is formed by cutting away a portion of the second oblate spheroid along a plane perpendicular to a minor axis of the second oblate spheroid.

The above features and advantages and other features and advantages of the present invention are readily apparent from the following detailed description of the best modes for carrying out the invention when taken in connection with the accompanying drawings.

### BRIEF DESCRIPTION OF THE SEVERAL VIEWS OF THE DRAWINGS

The present invention is illustrated by way of example, and not by way of limitation, in the figures of the accompanying drawings and in which like reference numerals refer to similar elements in the same figure. All the figures are schematic and generally only show parts which are necessary in order to elucidate the invention. For simplicity and clarity of illustration, elements shown in the figures and discussed below have not necessarily been drawn to scale. Well-known structures and devices are shown in simplified form, omitted, or merely suggested, in order to avoid unnecessarily obscuring the present invention.

FIG. 1 is a longitudinal central section of a massage and heat applicator containing a removable heating unit in the prior art.

FIG. 2 is a similar longitudinal section on a line at right angles to that of FIG. 1 in the prior art.

FIG. 3 is a longitudinal sectional view of the removable heating unit detached from the massage and heat applicator in the prior art.

FIG. 4 a fragmentary sectional view of the massage and heat applicator provided with a combined heating coil and thermostat in the prior art.

FIG. 5 is a schematic view of the thermal pillow apparatus in the prior art.

FIG. 6 is an isolated schematic view of a pillow element of the thermal pillow apparatus of FIG. 5 in the prior art.

FIG. 7A is a sectional view of a pillow element of the thermal pillow apparatus of FIG. 5 in the prior art.

FIG. 7B is a sectional view of another pillow element of the thermal pillow apparatus of FIG. 5 in the prior art.

FIG. 8 shows an axial section of a stroking-massage apparatus in the prior art on a plane.

FIG. 9 shows an axial section of the stroking-massage apparatus in FIG. 8 on a plane perpendicular to that in FIG. 8 in the prior art.

FIG. 10 illustrates some massaging devices in accordance with an exemplary embodiment of the present invention

FIG. 11 demonstrates the definition of a “truncated shape of oblate spheroid” in accordance with an exemplary embodiment of the present invention.

FIG. 12 is a perspective view of a constant hot temperature flat massage ball which is heated by a hot water bag in accordance with an exemplary embodiment of the present invention.

FIG. 13 is a perspective view of components of constant hot temperature flat massage ball which is heated by a hot water bag in accordance with an exemplary embodiment of the present invention.

FIG. 14 is a perspective view of constant hot temperature flat massage ball which is heated by an electric heating pad in accordance with an exemplary embodiment of the present invention.

FIG. 15 is a perspective view of components of constant hot temperature flat massage ball which is heated by an electric heating pad in accordance with an exemplary embodiment of the present invention.

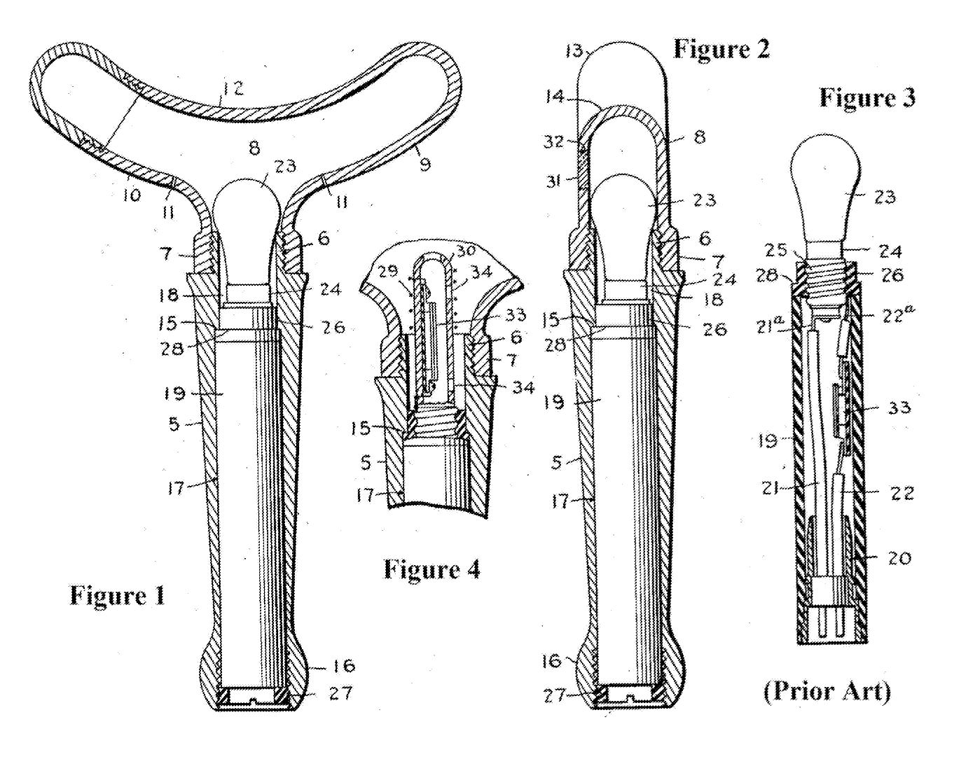

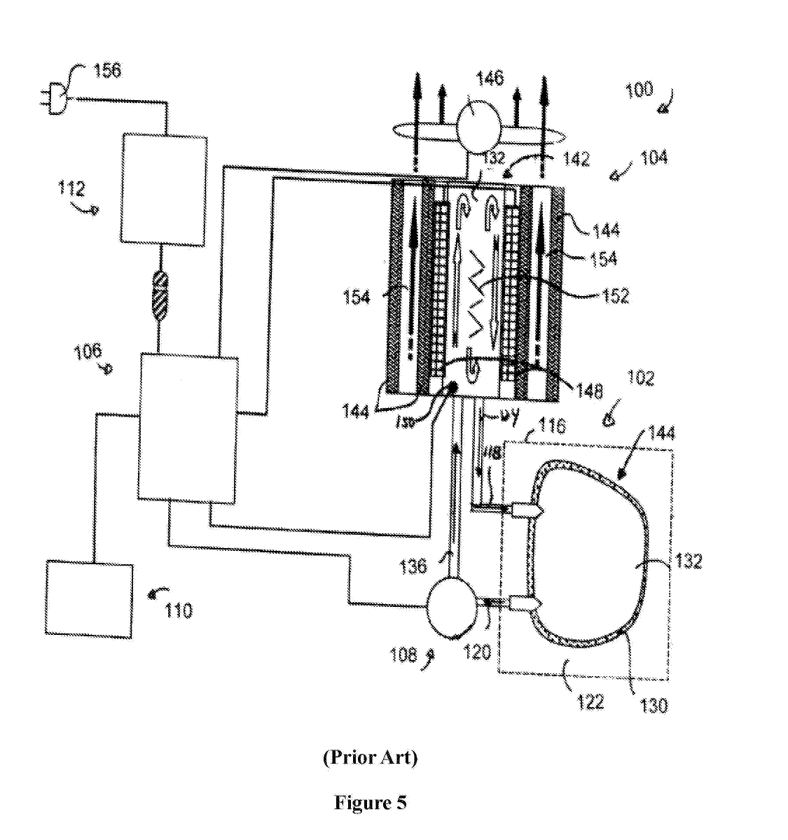

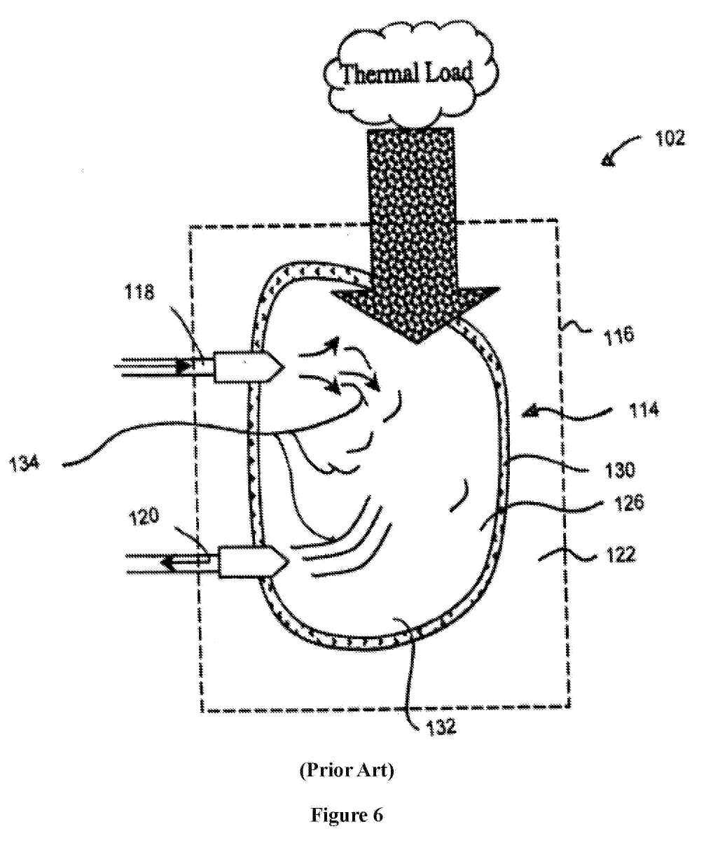

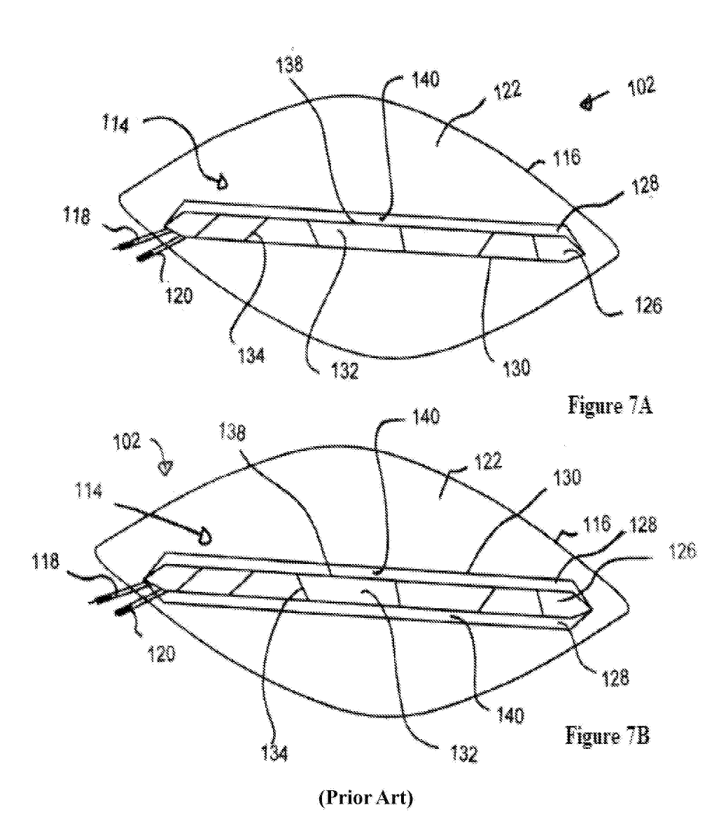

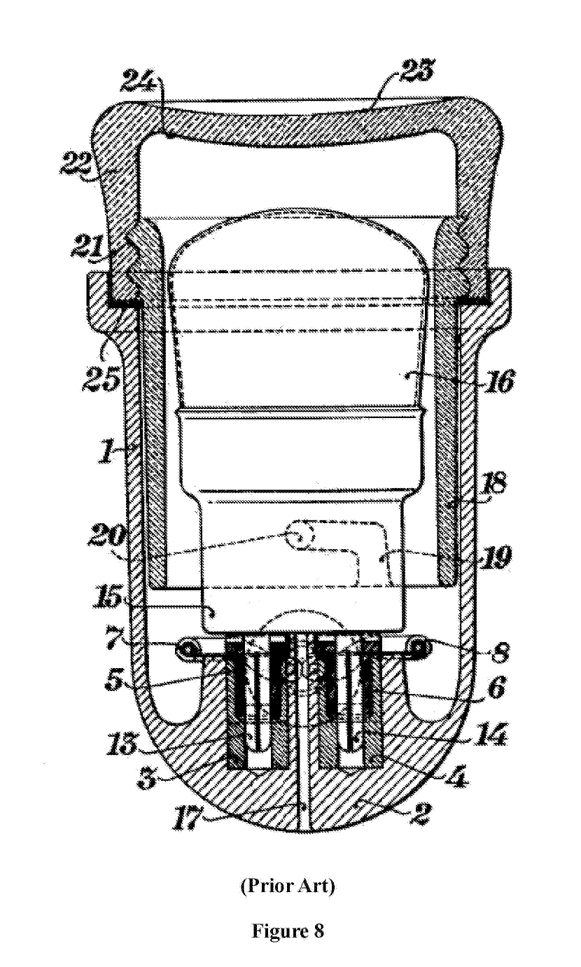

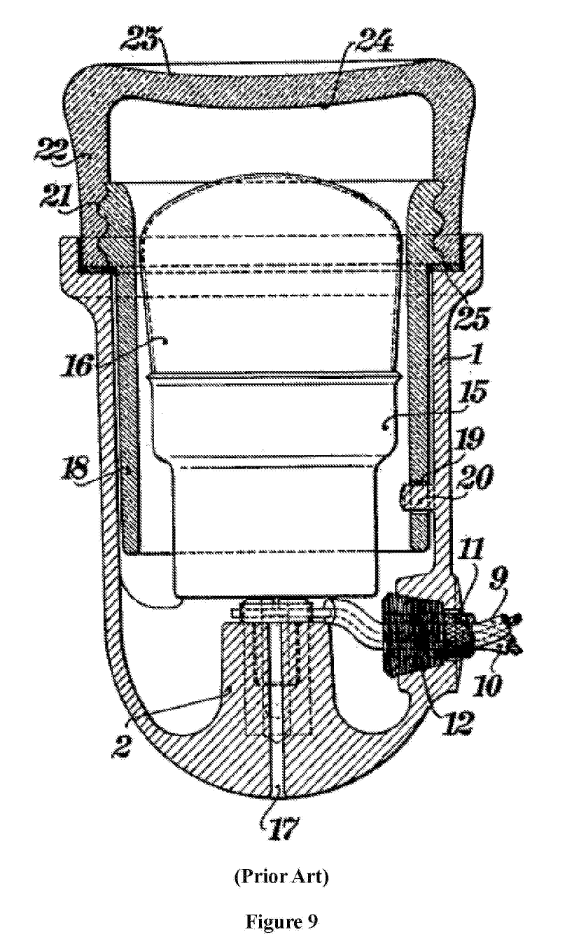

### DETAILED DESCRIPTION OF THE PREFERRED EMBODIMENT

In the following description, for the purposes of explanation, numerous specific details are set forth in order to provide a thorough understanding of the present invention. It is apparent, however, to one skilled in the art that the present invention may be practiced without these specific details or with an equivalent arrangement.

Where a numerical range is disclosed herein, unless otherwise specified, such range is continuous, inclusive of both the minimum and maximum values of the range as well as every value between such minimum and maximum values. Still further, where a range refers to integers, only the integers from the minimum value to and including the maximum value of such range are included. In addition, where multiple ranges are provided to describe a feature or characteristic, such ranges can be combined.

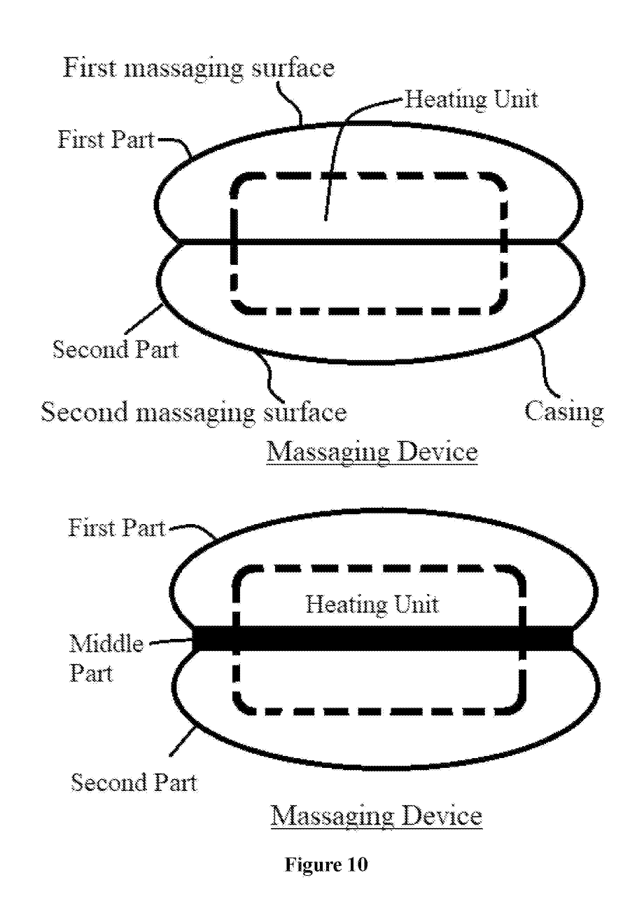

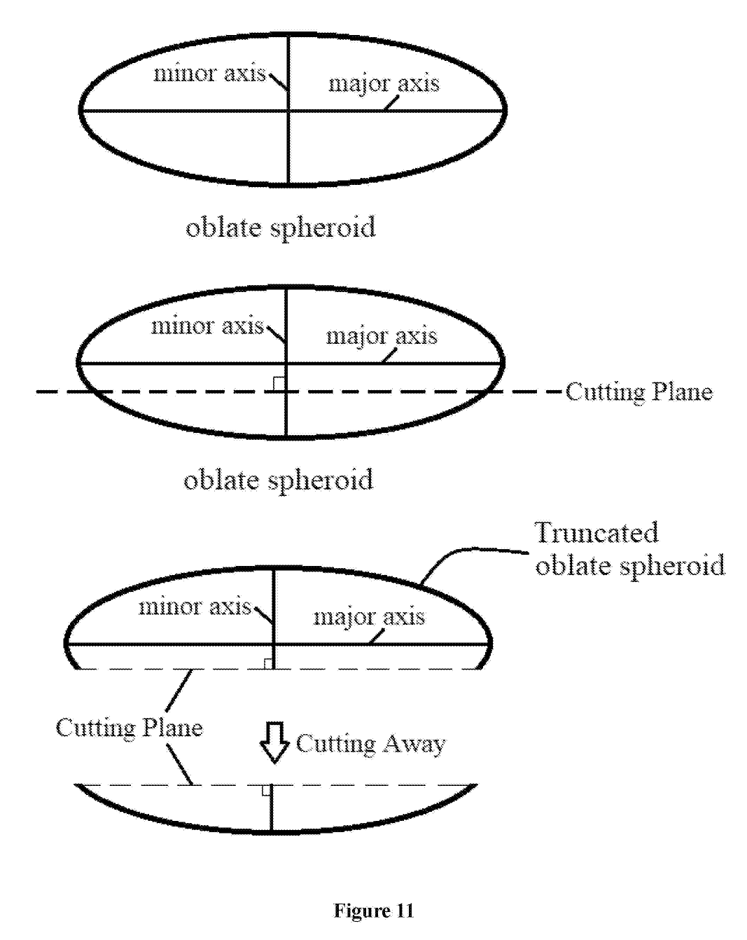

The massaging devices in FIG. 10 include two opposite massaging surfaces (a first massaging surface and a second massaging surface) for massaging a human body. When one of the two massaging surfaces is not as warm as desired, the user can flip the massaging device and use the other massaging surface on the human body. The user can keep flipping the massaging device as shown in FIG. 10 back and forth as many times as he/she wants.

The massaging devices in FIG. 10 include a casing and a heating unit encapsulated inside the casing The casing comprising a first part, a second part, and an optional middle part. A portion of or the entire, first part has a truncated shape of a first oblate spheroid. A portion of, or the entire, second part has a truncated shape of a second oblate spheroid.

The so-called “truncated shape” is explained with reference to FIG. 11 A spheroid, or ellipsoid of revolution, is a quadric surface obtained by rotating an ellipse about one of its principal axes. If the ellipse is rotated about its major axis, the result is a prolate (elongated) spheroid, shaped like an American football or rugby ball. If the ellipse is rotated about its minor axis, the result is an oblate (flattened) spheroid, shaped like a lentil, such as the first/second oblate spheroid as shown in FIG. 11. The truncated shape of the first oblate spheroid is formed by cutting away a portion of the first oblate spheroid along a plane perpendicular to a minor axis of the first oblate spheroid. The truncated shape of the second oblate spheroid is formed by cutting away a portion of the second oblate spheroid along a plane perpendicular to a minor axis of the second oblate spheroid.

The first oblate spheroid and the second oblate spheroid may have same size and shape (i.e. they are identical); alternatively they may have different size and shape. There is no specific limitation on the aspect ratio of the two oblate spheroids. For both the first oblate spheroid and the second oblate spheroid, the length along their major axis may be independently of each other at least 2 times longer than the length along their minor axis, for example 2-10 times longer or 3-8 times longer. Referring back to FIG. 11, the portion of the first oblate spheroid that has been cut away may be 5-95% (e.g. 45%, 50% or 55%) of the first oblate spheroid by volume. In a preferred embodiment, the portion of the first oblate spheroid that has been cut away is less than half (<50%) of the first oblate spheroid by volume. The portion of the second oblate spheroid that has been cut away may be 5-95% (e.g. 45%, 50% or 55%) of the second oblate spheroid by volume. In a preferred embodiment, the portion of the second oblate spheroid that has been cut away is less than half (<50%) of the second oblate spheroid by volume.

In an embodiment, the first oblate spheroid and the second oblate spheroid have the same size and shape (i.e they are identical), and the portion of the first oblate spheroid that has been cut away equals to the portion of the second oblate spheroid that has been cut away

Referring back to FIG. 10, the casing may further comprise a middle part located between the first part and the second part. There is no specific restriction on the size and shape of middle part, for example, the middle part may have a shape of right circular cylinder. For example, when (1) the first oblate spheroid and the second oblate spheroid have the same size and shape (i.e. they are identical), and (2) the portion of the first oblate spheroid that has been cut away equals to the portion of the second oblate spheroid that has been cut away, the middle part may have a shape of right circular cylinder with a diameter that equals to the diameter of the cross sectional circular face formed after a portion of the first/second oblate spheroid has been cut away. There are no specific limitations on the height of the right circular cylinder (or thickness of the middle part). The height may be for example 1-10 mm.

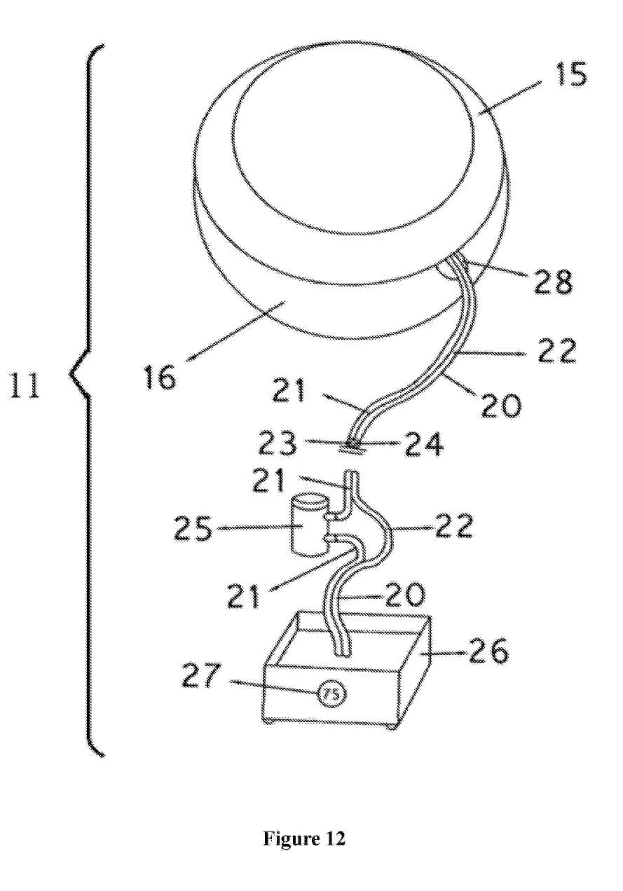

In exemplary embodiments as shown in FIGS. 12-15, the first part 15 of the massaging device has a top central recess 18 for enclosing the heating unit 19/31, and a female circular threaded wall 30 at an upper portion of the top central recess 18. The upper portion is proximal to the plane along which the first oblate spheroid is cut. The second part 16 of the massaging device has a bottom central recess 17 for enclosing the heating unit 19/31, and a male circular threaded embankment 29 extending around the bottom central recess 17. The male circular threaded embankment 29 is screwed into the female circular threaded wall 30. An opening or a groove 28 is built transversely across a wall of the bottom central recess 17 and the male circular threaded embankment 29.

In specific embodiments, the heating unit 19/31 may be a water bag 19, and the massaging device further comprises a connecting double-canal water tube, a one-way water pump, and a precision temperature hot water reservoir equipped with a thermostat. The groove 28 admits the water tube, and the water bag is stored inside the top central recess 18 and bottom central recess 17. The connecting double-canal water tube connects to the water bag at one end and one free end to be merged into the precision temperature hot water reservoir The water pump is installed in a middle portion of the double-canal water tube. Alternatively, the heating unit 19/31 may be an electric heating pad 31, and the massaging device further comprises one electric cord, one thermostat, and one plug. Similarly, the groove 28 admits the electric cord, and the electric heating pad is stored inside the top central recess 18 and bottom central recess 17 The electric cord connects the electric heating pad at one end and connects the plug at the other end. The thermostat is installed at a middle portion of the electric cord.

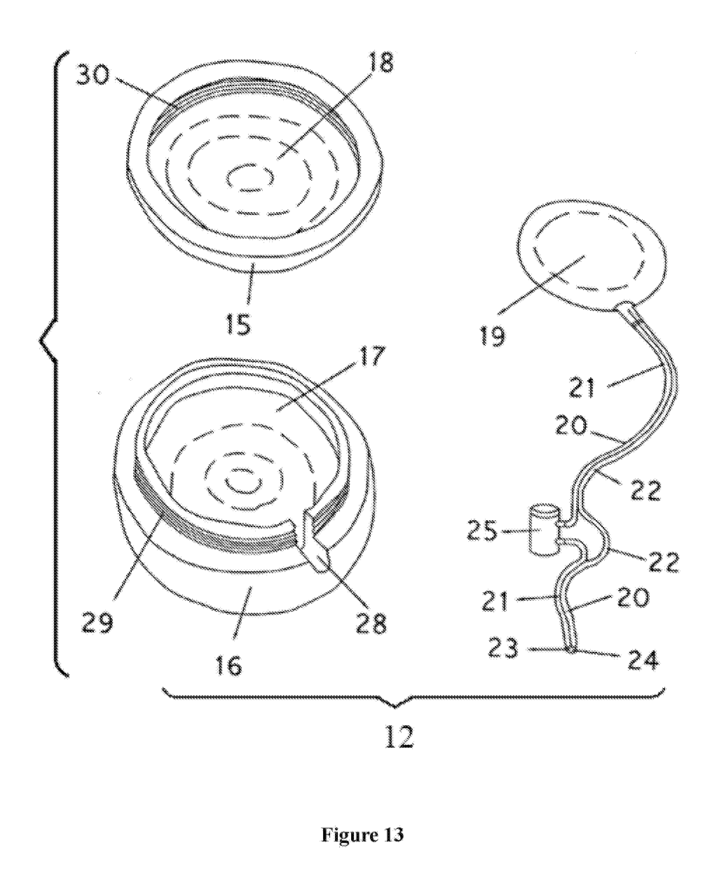

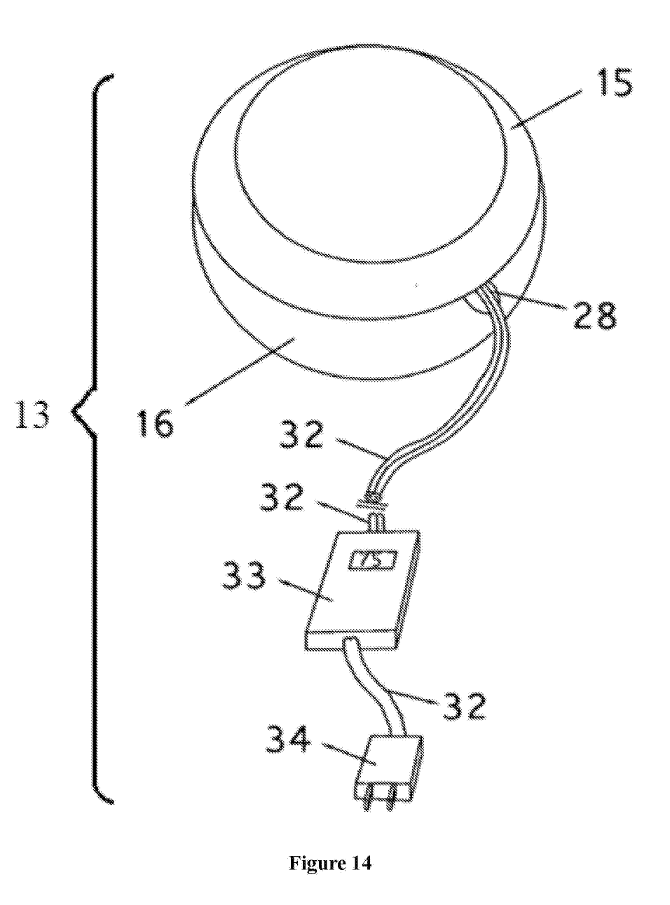

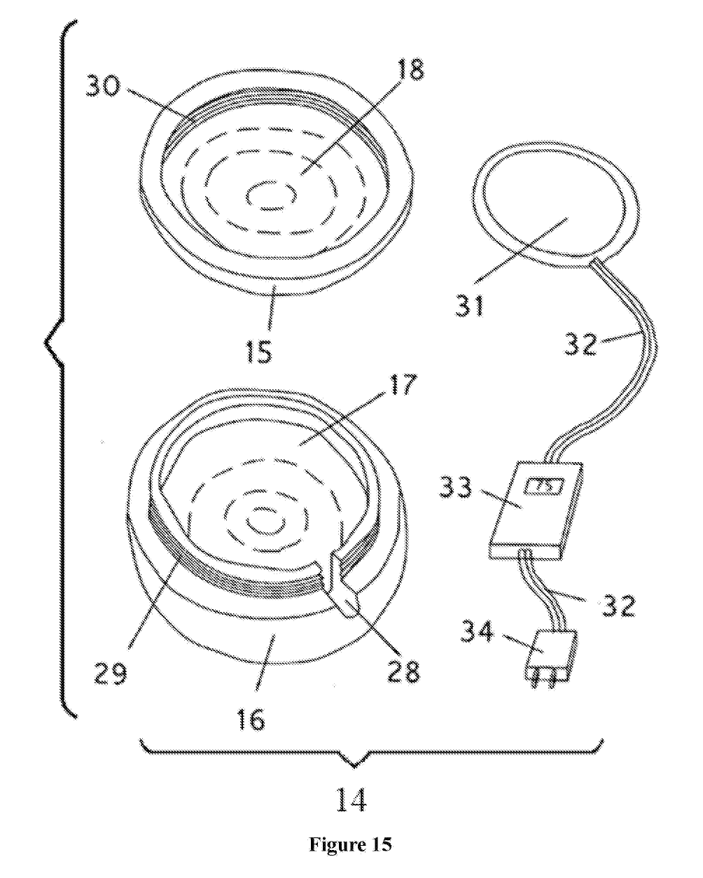

Referring now to FIGS. 12-15 for more details, the massaging device 11 in FIG. 12 includes a first part and a second part, both of which together constitute a constant hot temperature flat massage ball which shapes like a massage stone. The device 11 is heated by a heating unit such as a hot water bag, and device 11 is abbreviated as flat massage ball-HWB hereinafter. Components with reference number 12 of FIG. 13 are some components of flat massage ball-HWB 11. Similarly, the massaging device 13 in FIG. 14 is a constant hot temperature flat massage ball which shapes like a massage stone and is heated by an electric heating pad, and device 13 is abbreviate as flat massage ball-EHP hereinafter Components with reference number 14 of FIG. 15 are some components of flat massage ball-EHP 13.

Referring again to FIGS. 12-15 for more details, the first part 15 (or top shell-shaped receptacle 15) in FIGS. 12-15 includes a top shell-shaped receptacle of flat massager ball-HWB 11 and flat massage ball-EHP 13. The second part 16 (or bottom shell-shaped receptacle 16) in FIGS. 12-15 includes a bottom shell-shaped receptacle of massage flat ball-WHB and massage flat ball-EHP 13. Top shell-shaped receptacle 15 and bottom shell-shaped receptacle 16 can be made of gold or silver or copper or steel or aluminum or ceramic materials.

Component with reference number 17 in FIGS. 13 and 15 refers to a central recess of bottom shell-shaped receptacle 16. Component with reference number 18 is central recess of top shell-shaped receptacle 15. Component with reference number 19 of FIG. 13 is hot water bag. Hot water bag 19 is stored inside central recess 18 and central recess 17. Component with reference number 20 of FIGS. 12 and 13 is a water tube. Component with reference number 21 of FIGS. 12 and 13 is inward water tube of water tube 20. Component with reference number 22 of FIGS. 12 and 13 is outward water tube of water tube 20. Water tube 20 contains inward water tube 21 and outward water tube 22. Component with reference number 23 of FIG. 13 is canal of inward water tube 21. Component with reference number 24 of FIG. 13 is canal of outward water tube 22. Component with reference number 25 in FIGS. 12 and 13 is one-way water pump. Component with reference number 26 in FIG. 12 is precision temperature water reservoir. Component with reference number 27 in FIG. 12 is thermostat which is preferably adjusted at 100 to 104 degree Fahrenheit. Component with reference number 28 in FIGS. 12-15 is a groove of bottom shell-shaped receptacle 16. Groove 28 can admit water tube 20. Component with reference number 29 in FIGS. 13 and 15 is male threaded circular embankment of bottom shell-shaped receptacle 16. Male threaded circular embankment 29 is built around the central recess 17 as illustrated in FIGS. 13 and 15. Component with reference number 30 of in FIGS. 13 and 15 is female threaded circular wall. Female threaded circular wall 30 is built at upper portion of central recess 18. Male threaded circular embankment 29 can screw snugly and tightly into female threaded circular wall 30. When water pump 25 is turned on, it pumps hot water from precision temperature water reservoir 26 trough inward water tube 21 into hot water bag 19 rand then the cooler water returns from water bag 19 through outward water tube 22 into precision temperature hot water reservoir 26 to be reheated. Hot water bag 19 transmits heat to top shell-shaped receptacle 15 and bottom shell-shaped receptacle 16 to keep them at a constant hot temperature. The thermostat 25 is preferably adjusted at 100 to 104 degree Fahrenheit If hotter water temperature or lower temperature is desired the thermostat 27 of precision temperature water reservoir 26 can be adjusted to a desired higher temperature or lower temperature. Number 31 of FIG. 15 is an electric heating pad. Number 32 in FIGS. 14 and 15 is an electric cord. Number 33 in FIGS. 14 and 15 is a thermostat. Number 34 in FIGS. 14 and 15 is an electric plug. Electric heating pad 31 is stored inside central recess 17 and central recess 18. Electric cord 32 can pass through groove 28. After electric plug 34 is plugged in wall socket and the thermostat 33 is set at e.g. 100 degree Fahrenheit, the electric heating pad 31 starts to heat to reach a temperature of 100 degree Fahrenheit and transmits heat to top shell-shaped receptacle 15 and bottom shell-shaped receptacle 16 to keep them at a constant hot temperature at around 100 degree Fahrenheit.

The embodiments as shown in FIGS. 12-15 provide a constant hot temperature flat massage ball. The ball includes one top shell-shaped receptacle, one bottom shell-shaped receptacle and a central heating unit which is either a hot water bag, or an electric heating pad. The constant hot temperature flat massage ball is constantly heated by hat water bag or heating pad. Therefore, it can be used for continuously massage with massage body cream or lotion without interruption.

In the embodiments as shown in FIGS. 12-15, the top shell-shaped receptacle has a central recess to store the heating unit and a circular female threaded wall at the upper portion of central recess. The bottom shell-shaped receptacle also has a central recess to store the heating unit, a circular male threaded embankment above the central recess to screw into the above-mentioned circular female threaded wall and a groove which is built transversely across its wall and the above-mentioned male threaded embankment to admit the above mentioned circulating water tube and electric cord. The water bag of the above-mentioned central heating unit is heated by hot water, which is pre-heated to 100 to 104 degree Fahrenheit controlled by a thermostat in precision temperature hot water reservoir, through the circulating water tube pumped by a water pump. The central electric heating pad has a thermostat which is preferably adjusted at 100 to 104 degree Fahrenheit. The constant hot temperature flat massage ball is constantly heated by hat water bag or heating pad. Therefore, it can be used for continuously massage with massage body cream or lotion without interruption.

The presently invented constant hot temperature flat massage ball is kept at a constantly hot temperature either by a hot water bag or an electric heating pad at a fixed hot temperature, about 100 to 104 degree Fahrenheit. The conventional massage stones have to be removed after cooling off in two minutes and replaced by other heated massage stones.

In the foregoing specification, embodiments of the present invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicant to be the scope of the invention, is the literal and equivalent scope of the set of claims that issue from this application, in the specific form in which such claims issue, including any subsequent correction.

1. A massaging device having two opposite massaging surfaces for massaging human body, comprising a casing and a heating unit encapsulated inside the casing, wherein the casing comprising a first part including a truncated shape of a first oblate spheroid and a second part including a truncated shape of a second oblate spheroid, wherein the truncated shape of the first oblate spheroid is formed by cutting away a portion of the first oblate spheroid along a plane perpendicular to a minor axis of the first oblate spheroid, and wherein the truncated shape of the second oblate spheroid is formed by cutting away a portion of the second oblate spheroid along a plane perpendicular to a minor axis of the second oblate spheroid.

2. The massaging device according to claim 1, wherein the first oblate spheroid and the second oblate spheroid are the same.

3. The massaging device according to claim 2, wherein for both the first oblate spheroid and the second oblate spheroid, the length along their major axis is at least 2 times longer than the length along their minor axis.

4. The massaging device according to claim 3, wherein the portion of the first oblate spheroid that has been cut away is less than half of the first oblate spheroid, and wherein the portion of the second oblate spheroid that has been cut away is less than half of the first oblate spheroid.

5. The massaging device according to claim 4, wherein the portion of the first oblate spheroid that has been cut away equals to the portion of the second oblate spheroid that has been cut away.

6. The massaging device according to claim 5 wherein the casing further comprises a middle part located between said first part and said second part, and wherein said first part connects to said second part through the middle part.

7. The massaging device according to claim 6, wherein the middle part has a shape of right circular cylinder

8. The massaging device according to claim 1,
wherein the first part has a top central recess for enclosing the heating unit, and a female circular threaded wall at an upper portion of said top central recess, wherein said upper portion is proximal to the plane along which the first oblate spheroid is cut;
wherein the second part has a bottom central recess for enclosing the heating unit, and a male circular threaded embankment extending around said bottom central recess,
wherein said male circular threaded embankment is screwed into said female circular threaded wall, and
wherein a groove is built transversely across a wall of the bottom central recess and said male circular threaded embankment.

9. The massaging device according to claim 8, wherein the heating unit is a water bag, and the massaging device further comprises a connecting double-canal water tube, a one-way water pump, and a precision temperature hot water reservoir equipped with a thermostat;
wherein the groove admits the water tube;
wherein said water bag is stored inside said top central recess and bottom central recess;
wherein said connecting double-canal water tube connects to said water bag at one end and one free end to be merged into said precision temperature hot water reservoir; and
wherein said water pump is installed in a middle portion of said double-canal water tube.

10. The massaging device according to claim 8, wherein the heating unit is an electric heating pad, and the massaging device further comprises one electric cord, one thermostat, and one plug;
wherein the groove admits the electric cord;
wherein said electric heating pad is stored inside said'top central recess and bottom central recess;
wherein said electric cord connects said electric heating pad at one end and connects said plug at the other end; and
wherein said thermostat is installed at a middle portion of said electric cord
